(19) United States
(12) Patent Application Publication
(10) Pub. No.: US 2010/0215583 A1
(43) Pub. Date: Aug. 26, 2010

(54) CELL NUCLEUS-ENTERING COMPOSITIONS

(75) Inventors: Ewald HANNAPPEL, Uttenreuth (DE); Thomas HUFF, Erlangen (DE); Allan L. GOLDSTEIN, Washington, DC (US); David CROCKFORD, Newburyport, MA (US)

Correspondence Address: ROTHWELL, FIGG, ERNST & MANBECK, P.C., 1425 K STREET, N.W., SUITE 800, WASHINGTON, DC, 20005, US

(73) Assignees: REGENRX BIOPHARMACEUTICALS, INC., Bethesda, MD (US); UNIVERSITAET ERLANGEN-NUERNBERG, Erlangen (DE)

(21) Appl. No.: 12/632,654

(22) Filed: Dec. 7, 2009

Related U.S. Application Data
(62) Division of application No. 11/240,636, filed on Oct. 3, 2005.
(60) Provisional application No. 60/614,553, filed on Oct. 1, 2004.
(60) Provisional application No. 60/679,248, filed on May 10, 2005.
(60) Provisional application No. 60/684,993, filed on May 27, 2005.

(51) Int. Cl. A61K 49/00; A61K 38/08; A61K 38/16; A61P 35/00; A61P 43/00

(52) U.S. Cl. 424/9.1; 514/17; 514/16; 514/12

(57) ABSTRACT

A pharmaceutically acceptable composition and method for entering a cell nucleus utilizes a cell nucleus-entering polypeptide including at least one of amino acid sequence LKKTET (SEQ ID NO: 1), amino acid sequence LKKTNT (SEQ ID NO: 2) or amino acid sequence KSKLKK (SEQ ID NO: 3), or a conservative variant thereof, linked to a physiologically active agent having at least one of therapeutic or diagnostic application in the cell nucleus.

### CROSS-REFERENCE TO RELATED APPLICATIONS

The present application is a divisional of U.S. patent application Ser. No. 11/240,636, filed on Oct. 3, 2005, and claims the benefit of U.S. Provisional Application Ser. No. 60/614,553, filed Oct. 1, 2004, U.S. Provisional Application Ser. No. 60/679,248, filed May 10, 2005 and U.S. Provisional Application Ser. No. 60/684,993, filed May 27, 2005, the entire disclosures of which are incorporated herein by reference.

### BACKGROUND OF THE INVENTION

1. Field of the Invention

The present invention relates to the field of compositions and methods for delivering physiologically active agents.

2. Description of the Background Art

There is a need in the art for improved compositions and methods for delivering physiologically active agents.

### SUMMARY OF THE INVENTION

In accordance with the present invention, a pharmaceutically acceptable composition for entering a cell nucleus comprises a cell nucleus-entering polypeptide comprising at least one of amino acid sequence LKKTET (SEQ ID NO:1), amino acid sequence LKKTNT (SEQ ID NO:2) or amino acid sequence KSKLKK (SEQ ID NO:3), or a conservative variant thereof, linked to a physiologically active agent having at least one of therapeutic or diagnostic application in said cell nucleus.

### DETAILED DESCRIPTION OF THE INVENTION

The present invention provides compositions and methods utilizing actin-sequestering peptides such as thymosin β4 (Tβ4, TB4 or TBeta4) and other actin-sequestering peptides or peptide fragments containing amino acid sequence LKKTET (SEQ ID NO:1), LKKTNT (SEQ ID NO:2) or KSKLKK (SEQ ID NO:3), or conservative variants thereof. Included are N- or C-terminal variants such as KLKKTET (SEQ ID NO:4) and LKKTETQ (SEQ ID NO:5). These peptides and peptide fragments are useful for entering cell nuclei for treating and/or preventing various conditions, and affecting numerous physiological functions. In some preferred embodiments, the cell-entering peptide is Tβ4.

The physiologically active agent can be linked to the cell-entering peptide at any suitable position. For example, the agent can be linked to the N-terminus of Tβ4, or to an amino acid of the cell-entering peptide at another location, most preferably a glutamine residue. The agent can be a drug, chemotherapeutic agent, DNA sequence, RNA sequence, DNA- or RNA-activating or deactivating agent, diagnostic agent, or the like.

In preferred embodiments, the cell-entering peptide penetrates the nuclear membrane so as to carry the linked agent into the nucleus. The nuclear membrane preferably is mammalian, more preferably human.

The invention also is applicable to a method of administering an agent-carrying cell-entering peptide to a mammalian subject. The method may comprise contacting a nuclear membrane or tissue of a subject with a composition as defined herein, preferably a pharmaceutically acceptable composition. In preferred embodiments, the subject is mammalian, more preferably human.

Thymosin β4 initially was identified as a protein that is up-regulated during endothelial cell migration and differentiation in vitro. Thymosin β4 is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

Thymosin β4 is a member of the β-thymosin family of highly conserved polar 5-kDa polypeptides found in various tissues and cell types. Originally purified from thymus and regarded as a thymic hormone, thymosin β4 was then found to be involved in multiple biological processes. As the main G-actin sequestering peptide, it plays an important role in regulation of actin assembly during cell proliferation, migration, and differentiation. Numerous studies implicate thymosin β4 in regulation of cancerogenesis, inflammation, angiogenesis, and wound healing. It was found that thymosin β4 expression regulated tumorigenicity and metastatic activity in malignant cell lines through actin-based cytoskeletal organization. Thymosin β4 was found to be elevated in tube forming endothelial cells; it increases their attachment, spreading and migration thus promoting angiogenesis. Thymosin β4 was also found in ulcer extracts and wound fluids at high concentrations and was suggested to function as an antibacterial factor. The stimulating role of thymosin β4 in wound healing was demonstrated in several studies with animal models. When added topically or administered intraperitoneally, thymosin β4 enhanced dermal wound healing in a rat full thickness model. The ability to accelerate dermal wound healing has also been observed in db/db diabetic mice, steroid-immunosuppressed mice and in aged mice. Thymosin β4 has also been shown to accelerate healing of the corneal epithelium after burn injuries and to down regulate a number of corneal cytokines and chemokines reducing the inflammatory response.

Activation of the coagulation cascade upon vascular injury results in generation of thrombin which converts fibrinogen into fibrin. Fibrin polymerizes spontaneously to form blood clots which seals damaged places thus preventing the loss of blood. Fibrin also serves as a provisional matrix on which various cell types adhere, migrate and proliferate replacing fibrin with normal tissues during subsequent wound healing processes.

Thymosin β4 serves as a specific substrate for tissue transglutaminase and can be selectively cross-linked by it to collagen, actin, fibrinogen and fibrin, proteins which are also involved in the above mentioned processes. After activation of platelets with thrombin, thymosin β4 is released and cross-linked to fibrin in a time- and calcium-dependent manner.

In preferred embodiments, the cell-entering polypeptide comprises amino acid sequence LKKTET (SEQ ID NO:1), LKKTNT (SEQ ID NO:2), KSKLKK (SEQ ID NO:3), KLKKTET (SEQ ID NO:4), LKKTETQ (SEQ ID NO:5), Thymosin β4 (Tβ4), an N-terminal variant of Tβ4, a nucleus-entering C-terminal variant of Tβ4, an N-terminal fragment of Tβ4, an isoform of Tβ4, a splice-variant of Tβ4, oxidized Tβ4, Tβ4 sulfoxide, lymphoid Tβ4, pegylated Tβ4 or any other actin sequestering or bundling proteins having actin binding domains, or peptide fragments comprising or consisting essentially of the amino acid sequence LKKTET (SEQ ID NO:1), LKKTNT (SEQ ID NO:2) or KSKLKK (SEQ ID NO:3), or conservative variants thereof. International Application Serial No. PCT/US99/17282, incorporated herein by reference, discloses isoforms of Tβ4 which may be useful in accordance with the present invention as well as amino acid sequence LKKTET (SEQ ID NO:1), and conservative variants thereof, which may be utilized with the present invention. International Application Serial No. PCT/GB99/00833 (WO 99/49883), incorporated herein by reference, discloses oxidized Thymosin Γ4 which may be utilized in accordance with the present invention. Although the present invention is described primarily hereinafter with respect to TΓ4 and TΓ4 isoforms, it is to be understood that the following description is intended to be equally applicable to amino acid sequence LKKTET (SEQ ID NO:1), LKKTNT (SEQ ID NO:2), KSKLKK (SEQ ID NO:3), KLKKTET (SEQ ID NO:4), LKKTETQ (SEQ ID NO:5), peptides and fragments comprising or consisting essentially of LKKTET (SEQ ID NO:1), LKKTNT (SEQ ID NO:2), KSKLKK (SEQ ID NO:3), KLKKTET (SEQ ID NO:4), or LKKTETQ (SEQ ID NO:5), conservative variants thereof, as well as oxidized Thymosin β4, Tβ4 sulfoxide, lymphoid TΓ4 and pegylated Tβ4.

The cell-entering peptide with linked agent may be administered in any suitable effective amount. For example, the cell-entering peptide with linked agent may be administered in dosages within the range of about 0.1-50 micrograms, more preferably in amounts within the range of about 1-30 micrograms.

A composition in accordance with the present invention can be administered once, daily, every other day, every other week, every other month, etc., with a single application or multiple applications per day of administration, such as applications 2, 3, 4 or more times per day of administration.

TΓ4 isoforms have been identified and have about 70%, or about 75%, or about 80% or more homology to the known amino acid sequence of TΓ4. Such isoforms include, for example, TΓ4^{ala}, Tβ9, Tβ10, Tβ11, Tβ12, Tβ13, Tβ14 and Tβ15. Similar to TΓ4, the Tβ10 and Tβ15 isoforms, as well as the TΓ4 splice-variants, have been shown to sequester actin. TΓ4, Tβ10 and Tβ15, as well as other isoforms share an amino acid sequence, LKKTET (SEQ ID NO:1), that appears to be involved in mediating actin sequestration or binding. Although not wishing to be bound to any particular theory, the activity of Tβ4 isoforms may be due, in part, to the ability to regulate the polymerization of actin. β-thymosins appear to depolymerize F-actin by sequestering free G-actin. Tβ4's ability to modulate actin polymerization may therefore be due to all, or in part, its ability to bind to or sequester actin via the LKKTET (SEQ ID NO:1) sequence. Thus, as with Tβ4, other proteins which bind or sequester actin, or modulate actin polymerization, including Tβ4 isoforms having the amino acid sequence LKKTET (SEQ ID NO:1), are likely to be effective, alone or in a combination with Tβ4, as set forth herein, as are cell-entering peptides comprising sequence cell-entering.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms and Tβ4 splice-variants not yet identified, will be useful in the methods of the invention. As such Tβ4 isoforms are useful in the methods of the invention, including the methods practiced in a subject. The invention therefore further provides pharmaceutical compositions comprising agent-carrying Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET (SEQ ID NO:1), LKKTNT (SEQ ID NO:2) or KSKLKK (SEQ ID NO:3), for example, can similarly be employed in the methods of the invention. Such proteins include gelsolin, vitamin D binding protein (DBP), profilin, cofilin, adsevertin, propomyosin, fincilin, depactin, Dnasel, villin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, Dnasel, villin, fragmin, severin, capping protein, β-actinin and acumentin as set forth herein. Thus, the invention includes compositions and methods utilizing a polypeptide comprising the amino acid sequence LKKTET (SEQ ID NO:1), LKKTNT (SEQ ID NO:2) or KSKLKK (SEQ ID NO:3), (which may be within its primary amino acid sequence) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

The actual dosage, formulation or composition utilized may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable formulations may include the agent-carrying LKKTET (SEQ ID NO:1), LKKTNT (SEQ ID NO:2) or KSKLKK (SEQ ID NO:3) peptide in a carrier at a concentration within the range of about 0.0001-10% by weight, more preferably within the range of about 0.01-0.1% by weight, most preferably about 0.05% by weight. Any suitable pharmaceutically acceptable carrier may be utilized, such as water for injection.

The invention also relates to methods for delivering a physiologically active agent to a cell nucleus comprising administering to the cell nucleus a pharmaceutically acceptable composition as described herein. The method may involve administering the composition to a cell containing a nucleus, to a nucleus within a cell, to a mammalian subject, or by contacting tissue of a subject with the inventive composition.

### Example 1

Thymosin β4 is regarded as the main G-actin sequestering peptide in the cytoplasm of mammalian cells. It is also thought to be involved in cellular events like cancerogenesis, apoptosis, angiogenesis, blood coagulation and wound healing. Thymosin β4 has been previously reported to localise intracellularly to the cytoplasm as detected by immunofluorescence. It can be selectively labelled at two of its glutamine-residues with fluorescent Oregon Green cadaverine using transglutaminase; however, this labelling does not interfere with its interaction with G-actin. After microinjection into intact cells, fluorescently labelled thymosin β4 has a diffuse cytoplasmic and a pronounced nuclear staining. Enzymatic cleavage of fluorescently labelled thymosin β4 with AsnC-endoproteinase yielded two mono-labelled fragments of the peptide. After microinjection of these fragments, only the larger N-terminal fragment, containing the proposed actin-binding sequence exhibited nuclear localisation, whereas the smaller C-terminal fragment remained confined to the cytoplasm. In digitonin permeabilised and extracted cells, fluorescent thymosin β4 was solely localised within the cytoplasm, whereas it was found concentrated within the cell nuclei after an additional Triton X100 extraction. Thymosin β4 appears to be specifically translocated into the cell nucleus by an active transport mechanism, requiring an unidentified soluble cytoplasmic factor. This peptide may also serve as a G-actin sequestering peptide in the nucleus, although additional nuclear functions cannot be excluded.

Actin is present at high concentrations in virtually every eukaryotic cell. About half of the intracellular actin is stabilised in its monomeric form (G-actin) by interaction with sequestering factors. This monomeric actin can be used for the fast generation of new actin filaments after an appropriate intra- or extracellular signal. The β-thymosins constitute a family of highly conserved water soluble 5-kDa polypeptides. Thymosin β4 is the most abundant member of this family and is regarded as the main G-actin sequestering peptide in the cytoplasm of mammalian cells. This 43 amino acid oligopeptide forms a 1:1 complex with G-actin and thereby inhibits salt-induced polymerisation to F-actin. Additional members of the β-thymosin family have been identified and these peptides exhibit similar properties to thymosin β4. Thymosin β4 and other β-thymosins appear to be involved in a number of different processes like cancerogenesis and apoptosis. In the extracellular space, thymosin β4 participates in several physiological processes, e.g. angiogenesis, wound healing and regulation of inflammation. It also serves as a specific glutaminyl substrate of transglutaminases which crosslink thymosin β4 released from stimulated human platelets to fibrin and collagen.

There is increasing evidence for the presence of cytoskeletal proteins in the nucleus, such as actin itself, actin-related proteins (Arps) and a number of different actin binding proteins. Although the functions of these proteins in the nucleus are still under investigation, there is evidence that they are involved in activities ranging from nuclear assembly and shape changes to DNA replication and transcription. The intracellular localisation of thymosin β4 previously has never been studied in detail. One study using immunofluorescence described that its intracellular localisation in macrophages was most intense in the centre of the cell but was not nuclear. In another study, [¹²⁵I]-labelled thymosin β4 was injected into the cytoplasm of Xenopus laevis oocytes and the nuclear and cytoplasmic radioactivity was monitored. In these cells thymosin β4 was distributed roughly equally between cytoplasm and nucleus. The intracellular localisation of this peptide using a newly generated monospecific antibody against thymosin β4 was studied. Using the human mammary carcinoma MCF-7 cell line, variable cytoplasmic staining was found, and also additional nuclear staining.

Intracellular localisation by microinjecting fluorescently labelled thymosin β4 into cells of a number of different lines was studied. Thymosin β4 can be labelled at two of its three glutamine-residues by the enzymatic reaction of transglutaminase without influencing its G-actin sequestering activity. This technique was used to label thymosin β4 with Oregon Green cadaverine as a fluorescent marker. Fluorescence microscopic inspection after microinjection of the labelled peptide into cells of a number of different lines revealed that a considerable amount of thymosin β4 was located within their nuclei. The translocation of thymosin β4 into the nucleus is not achieved by simple diffusion, as the labelled peptide could not be detected within nuclei when the cells were previously treated with digitonin under conditions that extract the soluble components of the cytoplasm by permeabilisation of the plasma membrane while leaving the nuclear envelope intact. Nuclear localisation was observed only after subsequent treatment and permeabilisation of the nuclear membranes with Triton X100. These data are further supported by results showing that after enzymatic cleavage of bis-labelled thymosin β4 only the larger N-terminal fragment (Tβ¹⁻²⁶4), containing the proposed actin-binding site, was translocated to the nucleus. In contrast, the smaller C-terminal fragment (Tβ²⁷⁻⁴³4) and fluorescently labelled thymosin β4 chemically crosslinked to ADP-ribosylated actin were retained in the cytoplasm.

### Materials

Reagents were obtained from the following sources: LiChroprep RP18 (40-63 μm) and trifluoroacetic acid (Uvasol) from Merck (Darmstadt, Germany); guinea pig transglutaminase and 1-ethyl-3-[3-(dimethylamino)propyl]carbodiimide (EDC) from Sigma (Munich, Germany); AsnC-Proteinase from PanVera Corporation (Madison, Wis.); TRITC-phalloidin, Oregon Green-labelled deoxyribonuclease I and Oregon Green cadaverine were from Molecular Probes (Eugene, Oreg.).

### Protein Purification

Actin was prepared from rabbit skeletal or bovine heart muscle by the method of Pardee and Spudich and stored as G-actin in G-buffer (2 mM Tris, 0.2 mM ATP, 0.2 mM CaCl₂, 0.5 mM mercaptoethanol, 0.05% NaN₃, pH 8.0) at 0° C. Thymosin β4 was isolated from pig spleen as described. The purity of the preparation was demonstrated by reverse-phase HPLC. The concentrations of thymosin β4 and actin were determined by amino acid analysis after acid hydrolysis (6 M HCl, 155° C., 1 hour) and pre-column derivatisation with o-phthaldialdehyde/3-mercaptopropionic acid.

Fluorescently labelled thymosin β4 was prepared by incubation of 240 μg thymosin β4 (200 μM) with 120 μg Oregon Green cadaverine (OGC) (1 mM) and 0.2 U guinea pig transglutaminase at room temperature in 240 μl buffer consisting of 10 mM Tris-HCl, pH 7.4, 15 mM CaCl₂, 3 mM DTT. After 1 and 2 hours, 5 μl of the reaction mixture were subjected to HPLC analysis. The reaction was stopped after 4 hours by addition of 5 μl trifluoroacetic acid (TFA). Then the reaction mixture was subjected to preparative HPLC. Separated peptides were concentrated in vacuo and then characterised by amino acid analysis and MALDI-TOF mass spectrometry.

Proteolytic fragments of OGC-labelled thymosin β4 were prepared by the following procedure: 50 μg of peptide was incubated with 20 μU AsnC-endoproteinase in 100 μl reaction buffer (50 mM sodium acetate, pH 5.0, 0.2 mM DTT, 0.2 mM EDTA) for 16 hours at room temperature. Then the reaction was stopped by adding 5 μl 10% TFA and products were separated by preparative HPLC. Prior to analysis by MALDI-TOF-MS the samples were concentrated in vacuo.

In order to avoid intracellular dissociation of microinjected actin: thymosin β4 complex and/or its polymerisation, thymosin β4 was to ADP-ribosylated actin which is known to be polymerisation incompetent on its own chemically crosslinked. Therefore, fluorescently labelled thymosin β4 was crosslinked to ADP-ribosylated actin using EDC. ADP-ribosylated actin was generated by treatment of rabbit skeletal muscle actin with iota toxin and nicotinamide-adenosine dinucleotide (NAD).

Chromatographic conditions were controlled by a Merck-Hitachi L-6200 system supplemented with a diode-array UV detector (L-7450A, Merck-Hitachi), a reaction pump for post-column derivatisation (655A-13, Merck-Hitachi), and with a fluorometer (F-1050, Merck-Hitachi). The diode-array-detector signal was recorded on a computer using D-7000 HSM software (Merck) and the fluorescence signal on an integrator (D-2500, Merck-Hitachi). The flow rate was 0.75 ml/minute in 0.1% TFA (trifluoroacetic acid) with a 0-40% acetonitrile gradient over 60 minutes in a Beckman ODS Ultrasphere (5 μm, 4.6×250 mm) column. UV detection was at 205 nm and fluorescence was detected after post-column derivatisation with fluorescamine.

### Matrix-Assisted Laser Desorption Mass Spectrometry

Mass determinations were performed with a Biflex™ III MALDI-TOF mass spectrometer (Bruker Daltonics, Bremen, Germany). The instrument is equipped with a nitrogen laser (=337 nm) and a reflectron. Laser-desorbed positive ions were analysed after being accelerated by 19 kV in the reflection mode. External calibration was performed by use of a standard peptide mixture. Thirty individual spectra were averaged to produce a mass spectrum. Dried peptide samples were dissolved in 0.1% TFA containing 33% acetonitrile to a final concentration of about 20 ng/μl. Each sample (1 μl) was mixed with 2 μl of a saturated solution of α-cyano-4-hydroxy-cinnamic acid (Sigma, Germany) in 0.1% TFA, 33% acetonitrile and 1 μl of this mixture was spotted onto a stainless steel target.

### Determination of Dissociation Constants

Actin:thymosin β4 complex was generated by mixing equimolar concentrations of native or labelled thymosin β4 with G-actin. These mixtures were used for a comparative determination of dissociation constants of G-actin in complex with either thymosin β4 or its fluorescently labelled derivatives by equilibrium centrifugation.

### Viscometry

Viscometric measurements were done with a falling-ball viscometer at an angle of 40° relative to the horizontal over a distance of 45 mm with a 0.794 mm diameter ball. Forty-eight microliters G-actin solution (0.18 mg/ml in G-buffer) was incubated with or without thymosin β4 or Oregon Green cadaverine-labelled peptides for 15 minutes at room temperature and then 2 μl 50 mM MgCl₂ were added. The mixture was filled into a glass capillary (diameter 0.92 mm, 50 μl micro pipettes), sealed at one end and incubated for 4 hours before measuring.

### Generation of Polyclonal Antibody

A synthetic decapeptide representing the nine C-terminal amino acids of thymosin β4 with an additional cysteine residue at the N-terminus was conjugated to keyhole limpet hemocyanin (KLH, Sigma, Germany). New Zealand White rabbits were immunised with the KLH conjugate, with an amount corresponding to about 63 μg of the synthetic peptide emulsified with complete Freund's adjuvant (Sigma, Munich, Germany). Following a second immunisation, serum was collected and the anti-thymosin β4 antibody was partially purified from the serum by precipitation with 50% ammonium sulfate. The precipitate was dissolved in 5 mM phosphate buffer, pH 6.5, dialyzed against PBS and adsorbed with a 1% suspension of acetone powder from bovine heart in order to remove antibodies reacting non-specifically with cytoskeletal components. The antibody was affinity purified by passing it over a column coupled with immunogenic decapeptide. The resulting antiserum showed no cross-reactivity with other β-thymosins, actin, or any other cellular proteins of molecular weights in the range from 10-50 kDa as judged by western blot analysis and ELISA.

### Cell Culture

Kidney cells from African green monkey (Vero cells) and the human mammary cancer cell line MCF-7 were maintained in Dulbecco's MEM/F12 (Gibco, UK) supplemented with 10% (v/v) foetal calf serum (Gibco). The rat fibroblastic NRK, the human cervical cancer HeLa and epidermoid cancer A431 cell lines were maintained in DMEM supplemented with 10% FCS-gold (Invitrogen, Karlsruhe, Germany).

### Microinjection Experiments

Microinjection was performed with an ECET cell injection system (Eppendorf, Hamburg, Germany) consisting of the micromanipulator 5170 and the microinjector 5242 adapted to an Axiovert 100 inverted microscope (Zeiss, Göttingen, Germany). Microinjections were visually controlled by a CCD camera on a TV monitor (SSM 1210E, Sony, Tokyo, Japan). Fluorescent thymosin β4 and crosslinked ADP-ribosylated actin:thymosin β4 complex were injected into the cytoplasm at a concentration of 32 μM and 8.27 μM respectively, in 135 mM KCl, 5 mM Na₂HPO₄, pH 7.2. The injection pressure was between 65 and 80 hPa (1 hPa=0.1 kPa) and the injection time between 0.5 and 0.7 seconds.

### In Vitro Nuclear Translocation Experiments

HeLa cells were used for in vitro nuclear translocation experiments, as most previous nuclear translocation experiments have been performed with this cell line. HeLa cells were grown on coverslips until confluence was almost reached. Subsequently the cells were treated with slight modifications, i.e. they were washed three times with ice-cold PBS, placed on ice, and treated for 12 minutes with permeabilisation buffer (0.11 M potassium acetate, 5 mM magnesium acetate, 0.25 M sucrose, 0.5 mM EGTA and 20 mM HEPES, pH 7.5) supplemented with 40 μg/ml digitonin. Then the cells were washed three times with ice-cold permeabilisation buffer for 2, 5 and 10 minutes. In order to permeabilise the nuclear envelope, the digitonin extracted cells were incubated with 0.2% Triton X100 for 10 minutes and subsequently washed three times with permeabilisation buffer. Then the cells were incubated for 2 hours at room temperature with 3 μM fluorescent thymosin β4 in import buffer (120 mM potassium acetate, 5 mM magnesium acetate, 0.5 mM EGTA, 0.25 mM sucrose and 20 mM KHPO₄, pH 7.2).

### Fluorescence Microscopy

For immunofluorescence staining of MCF-7 cells with anti-thymosin β4 antibody cells were fixed with 1.2% paraformaldehyde, permeabilised with 0.2% Triton X100, washed in PBS and stained using anti-thymosin β4 antibody. Cell nuclei were counterstained by treating fixed cells with 1 μg/ml Hoechst 33258. Oregon Green labelled DNase I was employed to specifically stain monomeric actin. The microfilament system was visualised by staining paraformaldehyde fixed cells with TRITC-phalloidin as described (Paddenberg et al., 2001). Fluorescence microscopy and visualisation of microinjected fluorescent thymosin β4 was achieved either using standard or confocal microscopy with a Zeiss Axiophot equipped with epifluorescence optics, or with a Zeiss LSM510 confocal microscope (Zeiss, Göttingen, Germany).

Immunofluorescent Staining for Thymosin β4 in MCF-7 Cells Reveals Nuclear Localisation

In the course of the studies on the possible role of thymosin β4 in carcinogenesis were studied the intracellular localisation of this peptide in MCF-7 cells using an anti-thymosin β4 antibody in indirect immunofluorescence. In addition to the expected cytoplasmic localisation were detected a distinct staining of a number of nuclei as verified by their simultaneous staining with the chromatin specific dye Hoechst 33258. As the nuclear staining with anti-thymosin β4 varied, we also tested the possibility that there might have been an unequal distribution of monomeric actin in these cells. Therefore, the cells were counterstained with Oregon Green-labelled deoxyribonuclease I (DNase I), which is known to bind with high affinity and specificity to monomeric actin. Almost all nuclei were stained by the labelled DNase I indicating the presence of considerable amounts of G-actin in the nuclei of these cells. To further confirm the unexpected distribution of thymosin β4 its distribution was analysed with an alternative but more direct method. Therefore, microinjection of fluorescently labelled thymosin β4 was attempted.

Fluorescent Labelling of Thymosin β4 does not Influence its G-Actin Sequestering Activity

Oregon Green cadaverine (OGC)-labelled thymosin β4 was prepared as described in the experimental section. After preparative HPLC, isolated the two main products were showing Oregon Green fluorescence. Amino acid analysis of the isolated peptides showed identical amino acid compositions for both isolated products. The peptides were then characterised by mass spectroscopy. The data showed, that one of the labelled products was a mono-OGC [Tβ₄(OGC)₁] and the other was a bis-OGC thymosin β4 [Tβ₄(OGC)₂] derivative. Further analysis using enzymatic cleavage with AsnC-endoproteinase or trypsin and subsequent mass spectroscopy revealed that in the mono-labelled derivative Gln-36 and in the bis-labelled derivative Gln-23 and Gln-36 had been derivatised. For microinjection studies only the bis-labelled peptide was used.

To ensure that this labelling does not influence the G-actin sequestering activity the dissociation constant for the complex of bis-labelled thymosin β4 and bovine cardiac G-actin as well as its capacity to inhibit the salt-induced actin polymerisation were determined. Using equilibrium centrifugation, it was found that the dissociation constant for the complex of bis-labelled thymosin β4 with G-actin (0.47±0.1 μM) did not differ significantly from that determined for unlabelled thymosin β4 (0.59±0.08 μM). Inhibition of salt-induced actin polymerisation was tested using falling ball viscometry and showed inhibition of actin polymerisation by the labelled thymosin β4 at equimolar concentrations.

Microinjection of Labelled Thymosin β4 into Different Cell Lines Reveals Translocation into the Nucleus

To assay the intracellular distribution, bis-labelled thymosin β4 was microinjected into the cytoplasm of MCF-7 cells. As expected, directly after microinjection the labelled peptide was evenly distributed throughout the cytoplasm. After incubation for 1 hour a pronounced staining of the cell nucleus was detected. To ensure that the nuclear localisation was not a cell-specific artefact of the MCF-7 cells, microinjection experiments were also performed with Vero cells, where a comparable pattern was observed.

Next was analysed the distribution of fluorescently labelled intact thymosin β4 microinjected into additional cell lines. As in Vero and MCF-7 cells, thymosin β4 was also found within the nuclei of fibroblastic NIH-3T3 and NRK as well as the human epidermoid A431 cells after microinjection. One hour after microinjection, confocal sections of NIH-3T3 cells revealed a clear accumulation within the cell nucleus. Fixation of the microinjected cells after 30 minutes demonstrated nuclear accumulation together with varying fluorescence intensity within the cytoplasm. Three hours after microinjection was still observed a remaining although weaker cytoplasmic staining (data not shown). Within the nuclei we observed a homogenous distribution of the microinjected thymosin β4 with the exception that regions of presumed nucleoli were void of thymosin β4. A similar staining pattern was obtained with Oregon Green-labelled DNase I specific for G-actin.

In addition, microinjected NIH-3T3 or NRK cells was counterstained after paraformaldehyde fixation at various time points with TRITC-phalloidin to visualise actin filaments and to analyse the distribution of thymosin β4 in relation to the microfilament system. Besides its accumulation within the nucleus, the data obtained showed in many cases a faint punctuate staining along the cytoplasmic stress fibres of both fibroblastic NIH-3T3 and NRK cells together with a diffuse cytoplasmic localisation.

The immunohistochemical staining using the monospecific anti-thymosin β4 antibody had indicated in a number of cases that the nuclear staining was dependent on cell density, being more intense in isolated cells than in cells within cell clusters. Therefore, contacting A431 cells in the middle and periphery of cell clusters were microinjected. However, no dependence of the nuclear localisation of microinjected thymosin β4 on the intensity of their cell-cell contacts was found.

The low molecular mass of thymosin β4 (5 kDa) might suggest that it diffused through the nuclear pores by simple diffusion. To analyse its mode of nuclear translocation and accumulation, digitonin permeabilised and extracted HeLa cells was incubated with labelled thymosin β4. Digitonin treatment of cultured HeLa cells has recently been shown to permeabilise the plasma membrane for macromolecules, but to leave the nuclear envelopes structurally intact and competent for active transport. Fluorescence microscopy of HeLa cells thus treated revealed a solely cytoplasmic distribution of the labelled peptide, i.e. no staining of the cell nuclei. However, only after an additional treatment of the digitonin-extracted cells with 0.2% Triton X-100 for 10 minutes to also permeabilise the nuclear membranes was it possible to detect thymosin β4 within the nuclei. These data clearly indicate that the pore complexes of an intact nuclear envelope prevent the passage of thymosin β4 through the nuclear pores in the absence of soluble cytoplasmic factors. An identical mode of thymosin β4 exclusion was obtained with freshly isolated MCF-7 nuclei.

The N-terminal portion of thymosin β4 contains a sequence stretch enriched in lysine residues (¹⁴KSKLKK¹⁹ (SEQ ID NO:3)) suggestive of a functional nuclear localisation signal. As an initial test as to whether translocation into the nucleus depends on this basic sequence, bis-labelled thymosin β4 was digested using an AsnC-endoproteinase. Because thymosin β4 possesses only one asparagine residue at position 26, this digestion produced two fragments: an N-terminal fragment (thymosin β¹⁻²⁶4) and a C-terminal fragment (thymosin β²⁷⁻⁴³4) each bearing one fluorescent label. This was confirmed by HPLC analysis with detection of Oregon Green fluorescence. After isolation and characterisation of the labelled fragments by amino acid analysis and mass spectrometry, they were microinjected into Vero cells. The N-terminal fragment containing the ¹⁴KSKLKK¹⁹ (SEQ ID NO:3) sequence exhibited a pronounced nuclear localisation, whereas the C-terminal fragment was restricted to the cytoplasm.

The aforementioned sequence motif ¹⁴KSKLKK¹⁹ (SEQ ID NO:3) partially overlaps with the putative actin binding sequence of thymosin β4 (¹⁷LKKTET²² (SEQ ID NO:1)). To prove the assumption that the former motif is involved in nuclear translocation and to elucidate whether thymosin β4 is translocated into the nucleus in complex with actin, fluorescent actin:thymosin β4 complex was generated by chemical crosslinking with EDC. ADP-ribosylated rabbit skeletal muscle actin was used, as it has been previously shown to bind thymosin β4 but not to polymerise, in order to secure its monomeric state after microinjection. Successful crosslinking was verified by UV examination of the treated material after SDS-PAGE. Confocal microscopy of A431 cells after microinjection of the crosslinked complex showed that actin:thymosin β4 remained confined to the cytoplasm even after 3 hours.

### Discussion

It is now well accepted that thymosin β4 is the main G-actin sequestering peptide in the cytoplasm of mammalian cells. Together with other actin binding proteins that have F-actin severing and capping activities it is involved in the regulation of the ratio between monomeric (G-) and filamentous (F-) actin in the cytoplasm. Despite many publications dealing with the interaction of thymosin β4 with G-actin, there are few reports focussing on its intracellular localisation. In an early study the intracellular localisation of thymosin β4 was scrutinised by subcellular fractionation of rat spleen, and described to be mainly cytosolic with negligible amounts in the nuclear, mitochondrial/lysosomal or microsomal fractions. Subsequently the intracellular localisation of thymosin β4 and thymosin β10 in mouse peritoneal macrophages has been studied using specific antibodies raised against these two peptides. It has been reported that thymosin β4 immunofluorescence was most intense in the centre of the cell and lower in the periphery and the filopodia, with no staining of the nucleus. Because of its known function as a G-actin sequestering peptide, this cytoplasmic localisation seemed to be reasonable. More recently, the influence of polyamine depletion onto the actin cytoskeleton of migrating IEC-6 cells has been studied. These studies used a rabbit polyclonal antibody against thymosin β4 and described a primarily cytoplasmic staining in control cells, punctuate in appearance and close to or on the nuclear membrane, but no staining of the nucleus itself. In contrast, it was found that a prominent staining of the nucleus in polyamine-deprived cells or two minutes after treatment of control cells with epidermal growth factor. They concluded that the nuclear appearance resulted from a treatment-induced translocation of thymosin β4 into the nucleus. These results on thymosin β4 localisation in MCF-7 cells using indirect immunofluorescent labelling of an affinity-purified antibody indicate that cells with either cytoplasmic or nuclear accumulation can be found within a growing cell population. The differences between previous studies and these results showing variable degrees of cytoplasmic and nuclear localisation of thymosin β4 by using immunolocalisation may be caused by variations in the preservation of the original localisation of this highly soluble and diffusible peptide. Another possible explanation may be that recognition of thymosin β4 within the nucleus may depend on the epitope detected by the anti-peptide antibody. The polyclonal anti-peptide antibody used in this study recognises an epitope comprising of the four C-terminal amino acid residues of thymosin β4.

Recently, the nuclear localisation of actin itself, actin-related proteins (Arps) and a number of actin binding proteins has been reported. Although the functions of nuclear actin are far from being fully understood, it has been proposed that it might be involved in chromatin remodelling, mRNA processing and transport. It has been repeatedly reported that in contrast to the cytoplasm, the nuclei are devoid of phalloidin-stainable actin filaments. Indeed, it has been shown that a monoclonal antibody that presumably recognises a particular G-actin conformation yielded a punctuate nuclear staining pattern. Thymosin β4 was evenly distributed within the nucleus except for in nucleoli, which appeared to be free of this peptide. This distribution was observed after microinjection and immunostaining. This staining pattern coincided with the Oregon Green-DNase I staining and is suggestive of an intranuclear co-distribution of both thymosin β4 and G-actin. DNase I binds G-actin with high affinity and practically irreversibly. The nuclear staining and retention of Oregon Green-labelled DNase I was mainly due to actin binding, otherwise chromatin staining by Hoechst 33258 should have diminished or vanished owing to endonucleolytic activity of DNase I.

The fact that nuclear actin seems to be maintained in its monomeric form will necessitate G-actin sequestering factors inside the nucleus. However, the lack of phalloidin staining does not exclude the presence of special forms of F-actin in the nucleus. Indeed, cellular stress frequently induces the formation of nuclear actin rods that are composed of ADF/cofilin decorated actin filaments, which were shown not to bind phalloidin. In addition, it has also been proposed that F-actin may be present in the nucleus in the form of very short and/or highly branched filaments.

The data obtained by microinjection of fluorescent thymosin β4 into several different cell lines show a pronounced nuclear localisation of the labelled thymosin β4. After enzymatic digestion of bis-labelled thymosin β4 into two labelled fragments, only the larger N-terminal fragment, thymosin β¹⁻²⁶4, was translocated to the nucleus, whereas the smaller C-terminal fragment thymosin β²⁷⁻⁴³4 remained in the cytoplasm. The amino acid sequence of thymosin β4 does not contain a canonical nuclear localisation signal, but a cluster of positively charged amino acid residues (¹⁴KSKLKK¹⁹ (SEQ ID NO:3)) suggestive of a functional nuclear localisation signal, which partially overlaps with the proposed actin binding site of thymosin β4. Indeed analysis by SubLoc v1.0 predicted a nuclear localisation of intact thymosin β4 (accuracy 74%) as well as of its N-terminal fragment thymosin β¹⁻²⁶4 (accuracy 94%), whereas removal of just the above mentioned cluster from the amino acid sequence changes the prediction to cytoplasmic localisation (accuracy 95%). This assumption was further confirmed by the fact that chemically crosslinked actin:thymosin β4 complex, in which the ¹⁴KSKLKK¹⁹ (SEQ ID NO:3) motif might be sterically blocked by actin binding, is not translocated into the nucleus. Moreover, this result argues against a possible transport of thymosin β4 to the nucleus in complex with actin. The data suggest that this cluster of charged amino acid residues (¹⁴KSKLKK¹⁹ SEQ ID NO:3)) may be involved in the translocation of entire thymosin β4, as well as its N-terminal fragment into the nucleus.

As treatment of digitonin-permeabilised HeLa cells with labelled peptide resulted in a solely cytoplasmic localisation of the peptide, the translocation of thymosin β4 to the nucleus cannot be explained by a simple diffusion mechanism of the 5 kDa peptide, nor can it be caused by the fluorescent labelling. Therefore, it is proposed that thymosin β4 is translocated into the nucleus by an active transport mechanism, requiring an as yet unknown soluble cytoplasmic factor. It appears surprising that a peptide of only 5 kDa molecular mass does not freely diffuse through nuclear pores. However, structural studies have implicated that thymosin β4 is an elongated molecule and data from gel filtration experiments have shown that it migrates like a protein of considerably higher molecular mass.

The data clearly indicate that distinct amounts of the G-actin sequestering peptide thymosin β4 are translocated into the nucleus of cells by an active transport mechanism. When considered with the recent detection of G-actin and other actin binding proteins in the cell nucleus, the results suggest that thymosin β4 is not only the main G-actin sequestering peptide in the cytoplasm of mammalian cells, but may also account for the G-actin sequestering activity within the nucleus. As well as this presumed nuclear activity of thymosin β4 t is tempting to speculate that it may have additional functions in the nucleus as also proposed for actin itself and other actin binding proteins.

Alternatively, a specific function for nuclear actin has been questioned as it was shown that actin itself contains two nuclear export sequence stretches. Indeed, it has also been shown that nuclear accumulation of the actin binding protein profilin only serves to facilitate its nuclear export. In view of these data, it might also be possible that the nuclear accumulation of thymosin β4 stabilises actin in its monomeric state by forming a G-actin:thymosin β4 complex that is subsequently transformed into a G-actin:profilin complex (as during actin cycling in the cytoplasm) ready for nuclear export. Thus the supposed sequestering activity of thymosin β4 within the nucleus might also support the process of nuclear export of actin, which probably occurs after mixing cytoplasmic and nuclear contents during the open mitosis of mammalian cells.

### Example 2

The Beta-thymosins constitute a family of highly conserved 5 kDa peptides that are present in many tissues and almost every cell of various vertebrates and invertebrates. Thymosin Beta4 (TBeta4), the most abundant member of this peptide family in mammalian cells, is now regarded to be the main intracellular G-actin sequestering peptide. This 43-amino acid oligopeptide forms a 1:1 complex with G-actin, and, thereby, inhibits salt-induced polymerization to F-actin. All other tested members of this peptide family exhibit the same G-actin-sequestering activity, forming complexes. Members of this peptide family are also involved in carcinogenesis and metastasis. It has been shown that they are increasingly expressed in metastatic tumors of the prostate, breast, and thyroid. Treatment of breast cancer cells with chemotherapeutic drugs results in decreased expression of Beta-thymosins.

Beside its important intracellular function as a G-actin-sequestering peptide, there is increasing evidence for additional, probably extracellular functions of TBeta4.

Extracellular TBeta4 may contribute to physiological processes like angiogenesis, wound healing, and regulation of inflammation. This peptide increases the rate of attachment and spreading of endothelial cells, stimulates migration of human umbilical vein endothelial cells, promotes aortic ring vessel sprouting, induces matrix metalloproteinases, markedly accelerates healing of the skin and corneal wounds, and modulates a number of inflammatory cytokines and chemokines. TBeta4 is present in most tissues and cells of mammals, and is found in particularly high concentrations in blood platelets, neutrophils, macrophages, and lymphoid cells. But, as it does not possess a signal sequence for secretion, its concentration in plasma is low. However, under certain conditions (e.g., clotting), levels in serum can increase substantially, as it has been shown that this peptide is released from thrombin-stimulated blood platelets and attached to fibrin and collagen by factor XIIIa.

Additionally, TBeta4 has been suggested to be the precursor of the tetrapeptide, acSDKP (SEQ ID NO:6), the N-terminal sequence of TBeta4, that can be generated by a single cleavage step employing either a prolyl endopeptidase or an AspN-like protease. AcSDKP (SEQ ID NO:6), which was initially purified from fetal calf bone marrow and later chemically synthesized, as well as TBeta4 are known as negative controllers of normal hematopoiesis.

Mast cells derive from undifferentiated hematopoietic precursor cells and mature in the peripheral tissues as a resident cell. This peripheral maturation determines the heterogeneity of mast cell populations (e.g., differences in phenotype, reactivity to agonist stimuli, granular content, secretion patterns, etc.).

Mast cells are ubiquitous in the connective tissues and mucous membranes, especially in interface tissues (e.g., skin, respiratory tract, gastrointestinal mucosa) and are known to release, by means of degranulation, essential mediators to trigger inflammation and wound healing after an appropriate stimulus.

To further elucidate a possible role of TBeta4 and acSDKP (SEQ ID NO:6) as inhibitors of cell proliferation, it was studied whether TBeta4 and/or the tetrapeptide acSDKP (SEQ ID NO:6), might directly affect proliferation of bone-marrow-derived mast cells (BMDMCs). Additionally, to gain better insight as to how these peptides might modulate inflammatory responses and wound healing, it was also examined their effect on degranulation of peritoneal mast cells. Both peptides inhibit mast cell proliferation and induce degranulation in a concentration-dependent manner. As part of these studies, it was also found that both peptides induce an unusual non-apoptotic nuclear dysplasia in BMDMCs. Results. TBeta4 and acSDKP (SEQ ID NO:6) Inhibit Proliferation of Murine Bone-Marrow-Derived Mast Cells. Significant inhibition of proliferation was observed in BMDMCs exposed for six days to various concentrations of either TBeta4 or acSDKP (SEQ ID NO:6). Inhibition could be detected at all concentrations between 10⁻¹⁴ to 10⁻¹⁷ M with the maximum effect at 10⁻¹⁴ M. AcSDKP (SEQ ID NO:6) seemed to be a somewhat more potent inhibitor of proliferation than TBeta4.

TBeta4 and acSDKP (SEQ ID NO:6) Induce Dysplastic Nuclei in Cultured Mast Cells. BMDMCs treated with TBeta4 or acSDKP (SEQ ID NO:6) showed an unusual dysplastic appearance of the nuclei when compared to untreated cells. To confirm that dysplastic cell compartments were really nuclear components, cells were also stained with DAPI. Selected tryptic fragments of TBeta4 were tested, which contain neither the N-terminal tetrapeptide nor the proposed actin-binding sequence, as well as amino acid mixtures resulting from complete acid hydrolysis of TBeta4, and no dysplastic mast cell nuclei were observed. In addition, the effect of another tetrapeptide, Ac-Ser-Gln-Asn-Tyr (acSQNY (SEQ ID NO:7)) on BMDMCs was investigated, but no comparable dysplastic nuclei were found. To determine if TBeta4 and acSDKP (SEQ ID NO:6) treatment would cause dysplastic nuclei in immortal mast cells, we treated a C57 mast cell line for 6 days with 10⁻⁸, 10⁻¹², 10⁻¹⁴ or 10⁻¹⁹M. TBeta4 or acSDKP (SEQ ID NO:6). Only a few dysplastic nuclei were found when the cells were stained with either toluidine blue or May-Gruenwald-Giemsa solution.

### Dysplastic Nuclei are not Due to Apoptosis.

To further elucidate if the dysplastic nuclei were due to apoptosis, BMDMCs were treated with either TBeta4 or acSDKP (SEQ ID NO:6) and examined using a TUNEL assay. Based on this technique, none of the nuclear bodies in the treated mast cells were found to be stained by the TUNEL assay for apoptosis, whereas the positive control, BMDMCs treated with etoposide VP16, stained strongly by this assay. These results indicated that the dysplastic nuclei induced by TBeta4 and acSDKP (SEQ ID NO:6) were not due to apoptosis.

TBeta4 or acSDKP (SEQ ID NO:6)-Treated Mast Cells Are Not Multinucleated but G2-Growth-Arrested. It was determined whether the dysplastic nuclei were due to polyploidy or growth arrest during the cell cycle of the mast cells. BMDMCs exposed to either TBeta4 or acSDKP (SEQ ID NO:6) were stained with PI and analyzed by flow cytometry. The results revealed an accumulation of treated cells in G2 growth arrest.

Mast-Cell Degranulation Occurs in Response to TBeta4 or acSDKP (SEQ ID NO:6). To examine the effect of TBeta4 and acSDKP (SEQ ID NO:6) on mast cell degranulation, peritoneal mast cells co-cultured overnight on 3T3 fibroblast monolayers were treated for 2 h with either 10⁻⁸ or 10⁻¹⁴M TBeta4 or acSDKP (SEQ ID NO:6). Mast-cell degranulation was demonstrated by staining the cultures with toluidine blue. This analysis revealed exocytosis of mast cell granules in cells treated with either TBeta4 or acSDKP (SEQ ID NO:6) when compared to the negative control. At both concentrations tested, acSDKP (SEQ ID NO:6) caused significantly more degranulation than TBeta4 (P<0.003).

### Discussion

In this study, it was demonstrated that both TBeta4 and its N-terminal tetrapeptide, acetyl-N-Ser-Asp-Lys-Pro (acSDKP (SEQ ID NO:6)), inhibit proliferation of BMDMCs in a concentration-dependent manner. It was also shown that both peptides induce an unusual non-apoptotic nuclear dysplasia. In addition, it was established that both peptides induce degranulation of mast cells that are found in the peritoneal cavity.

On a molar basis, the tetrapeptide appears to be more biologically active than the protein.

At low concentrations, binding at the high-affinity site inhibits proliferation, but as the concentration increases, binding at the low-affinity site commences to offset more and more of the inhibition. However, as TBeta4 is exemplary of small, acidic peptides that have multiple physiological functions, a bell-shaped curve of inhibition of proliferation might well be the result of the competing nature of two or more of these functions. Future studies are needed to elucidate the mechanism of the functions of this important regulatory peptide that has been implicated to play a role in carcinogenesis, metastasis, chemotherapy, etc.

Experiments designed to show that dysplastic nuclei were specific to mast cells newly derived from the bone marrow were successful: peptide treatment of a mast cell line failed to cause nuclear dysplasia. Neither treatment with another tetrapeptide, acetyl-Ser-Gln-Asn-Tyr (acSQNY (SEQ ID NO:7)), nor amino acid mixtures resulting from complete acid hydrolysis of TBeta4 produced cells with dysplasic nuclei. Tryptic fragments of TBeta4, which neither contain the N-terminal tetrapeptide nor the proposed actin-binding sequence did not induce dysplastic nuclei, implying that the N-terminal fragment and possibly actin play a role in the dysplasia. Flow cytometric analysis of ploidy revealed that the dysplastic nuclei were not multi-nucleated. This is the first report of acSDKP (SEQ ID NO:6)-induced inhibition of cells in G2 phase, although it has been reported that acSDKP (SEQ ID NO:6) affected cells in G0 or early G1. This is also the first report showing that TBeta4 and acSDKP (SEQ ID NO:6) induce dysplastic nuclei in mast cells without inducing mast cell apoptosis suggesting a new way to generate G2-arrested mast cells for cell-cycle study.

In recent years, several reports have shown that TBeta4 may be involved in a number of cellular processes including regulation of inflammation, angiogenesis, and wound healing. For example, it was found that TBeta4 mRNA increases fivefold during the morphological differentiation of endothelial cells into capillary-like tubes. Furthermore, transfection of these cells with TBeta4 caused an increased rate of attachment and spreading on matrix components, and an accelerated rate of tube formation on Matrigel. It was also demonstrated that TBeta4 stimulates the migration of human umbilical vein endothelial cells, accelerates skin wound healing, and induces matrix metalloproteinase 2 in vitro and in vivo. Additionally, TBeta4 had been shown to promote corneal wound healing and to modulate the inflammatory response. Recently, it has been shown that TBeta4 serves as a specific substrate of transglutaminase, and that it is released from activated platelets and cross-linked to fibrin and collagen by factor XIIIa, providing a mechanism to increase the normally low concentration of this peptide near sites of clots and tissue damage. Here, we present further insight as to how TBeta4 and acSDKP (SEQ ID NO:6) may contribute to modulation of the inflammatory response and wound healing by showing that these peptides induce degranulation of mast cells that are found in the peritoneal cavity, and that have matured naturally in vivo, and are free to migrate to sites of tissue damage. Mast cell granules contain several biochemical mediators (e.g., histamine, protease, chymase, tumor necrosis factor) that are released by means of degranulation, after an appropriate stimulus. These released mediators are essential to trigger the different phases (inflammation, proliferation, and remodeling) of wound healing. Based on these data, it is tempting to speculate that TBeta4 is involved in this regulation by triggering degranulation of mast cells.

In spite of the increasing number of publications dealing with the multiple physiological effects of TBeta4, especially the extracellular effects, little is known about the pathways responsible for transducing these effects. To identify these pathways will be a pivotal point of future research in this field.

### Experimental Part

### Thymosin Beta4 and Tetrapeptides.

Thymosin Beta4 (TBeta4) was purified from pig spleen. Tryptic fragments of TBeta4 were prepared by incubating 100 μg of the peptide with 1 μg of sequencing-grade trypsin (Boehringer Mannheim, D-Mannheim) for 16 h. Thereafter, the proteolytic fragments were separated by reversed-phase HPLC and analyzed by MALDI-TOF-MS and amino acid analysis. Fragments containing neither the N-terminal tetrapeptide nor the actin-binding sequence were then remixed. Complete amino acid hydrolysis of TBeta4 was achieved by hydrolyzing the peptide for 1 h at 155° C. in 6M HCl. The tetrapeptides acetyl-NSer-Asp-Lys-Pro (acSDKP (SEQ ID NO:6)) (Mr 487) and acetyl-Ser-Gln-Asn-Tyr (acSQNY (SEQ ID NO:7)) (Mr 552.5) were purchased from Sigma Chemical Co., St. Louis, Mo.). Bone-Marrow-Derived Mast-Cell (BMDMC) Cultures. Bone-marrow plugs were isolated from normal BALB/c mice femurs. After lysis of erythrocytes, cells were resuspended at 2×105 cells/ml in complete DMEM (cDMEM): Dulbecco's Modified Eagle Medium (DMEM; Gibco BRL) containing 10% fetal bovine serum (FBS; Biowhittaker), 1.2% HEPES buffer, 2 mM L-glutamine, 100 U/ml penicillin, 100 U/ml streptomycin (Gibco BRL) supplemented with 50 ng/ml recombinant mouse stem cell factor and 100 U/ml IL-3 (Biosource).

The cells were cultured at 37° C. in a humidified atmosphere containing 5% CO₂ and 5% O₂ for at least 17 days to obtain bone-marrow-derived mast cells.

### Proliferation Assay and Morphology Determination

Cultured BMDMCs were plated in cDMEM in 12-well tissue culture plates at a concentration of 4×10⁴ cells/ml/well. The cells were left untreated or treated with daily additions of TBeta4 or AcSDKP (SEQ ID NO:6) at final concentrations ranging from 10⁻⁶ to 10⁻²² M for 6 days. The cells from each well were then harvested for cytospins or resuspended in 150 μl of cDMEM, followed by plating 50 μl of each into 3 wells of a 96-well plate. [³H] Thymidine (1 pCi/well) was added, and the cultures were further incubated for 26 h. The cells were harvested onto microplate filters using the harvester system of Packard FilterMate™. The amount of thymidine incorporation was determined with a microplate scintillation counter (TopCount™, Packard A Canberra Co). The morphology of cells was determined by staining cytospin preparations with toluidine blue. Mast cell nuclei features were assessed by staining with 4′,6′-diamidino-2-phenylindole dihydrochloride (DAPI; Boehringer Mannheim GmbH, Germany). Staining was for 10 min in the dark with 10 μg/ml DAPI in MeOH, followed by a MeOH rinse and air drying. Cover slips were placed on the slides using 5% N-propylgallate (Sigma Chemical Company, St. Louis, Mo.), and the cells were visualized under a fluorescent microscope (Olympus Provis).

### TUNEL Assay for Apoptosis.

Cytospin preparations of BMDMCs untreated or treated with either TBeta4 or AcSDKP (SEQ ID NO:6) were fixed in 4% paraformaldehyde for 10 min at r.t. The positive control consisted of cells treated with 100 μM of etoposide (VP16) for 18 h at 37° C. The cells were then permeabilized with acetone/EtOH 1:2 at −20° C. for 5 min, followed by rinsing twice with PBS, and air-dried. To block non-specific binding, the slides were treated with filtered BSA (bovine serum albumin; 1 mg/ml) in PBS (phosphate-buffered saline) for 10 min at r.t. The TUNEL assay was then carried out according to manufacturer manual (Boehringer Mannheim GmbH, Germany) with some modifications. Briefly, the assay was performed by incubating the slides in the dark at 37° C. for 1 h in a staining mixture of 4 μl of buffer, 0.2 μl of terminal transferase, 2 μl of CoCl₂, 0.4 μl of fluorescein-12-dUTP, and 13.4 μl of glass-distilled H₂O. After incubation, the cytospins were washed twice with PBS, air-dried, and covered with 5% N-propyl gallate in 70% glycerol/PBS, followed by sealing the cover slips with fingernail polish. The cells were then visualized with a fluorescent microscope.

### Flow-Cytometric Analysis of Ploidy

BMDMCs (10⁻⁶) untreated or treated with either 10⁻¹² M TBeta4 or acSDKP (SEQ ID NO:6) were re-suspended in 1 ml of fluorochrome staining soln. containing 50 μg/ml of propidium iodide (PI), 3.8 M sodium nitrate, 0.1% Triton X-100, and 1 U/ml RNAse B. The cells were then incubated on ice for 1 h, and data were acquired with an ELITE Flow cytometer.

### Antibodies

2.4G2, a monoclonal antibody that blocks binding to FcεRII/III, and B3B4, a monoclonal antibody that blocks binding to FcεRII, were isolated from ascitic fluid. Anti-IgE-DNP was purchased from Pharmingen, San Diego, Calif.

### Mast-Cell Degranulation Assay

Peritoneal mast cells from Balb/c mice were isolated with a 72.5% Percoll gradient and plated on top of a 3T3 (ATTC, Bethesda, Md.) fibroblast confluent monolayer in cDMEM at 37° C. overnight. The co-cultures were then left untreated or treated with either TBeta4 or acSDKP (SEQ ID NO:6) at concentrations of 10⁻⁸ and 10⁻¹⁴ M for 2 h at 37° C. Anti-IgE-DNP and DNP-HSA were added to one group of co-cultures for a positive degranulation control. After air-drying of the monolayer co-cultures, the cells were stained with toluidine blue at pH 2.8 for microscopic examination. The frequency of degranulated cells was determined by counting the number of degranulated cells and the total number of cells in the monolayer dish.

### Example 3

Physiologically active agents having therapeutic and/or diagnostic application in a cell nucleus are linked to T64 as follows. The agents are selected from drugs, chemotherapeutic agents, DNA sequences, RNA sequences, DNA- or RNA-activity or deactivity agents and diagnostic agents.

Agent-linked thymosin β4 is prepared by incubation of 240 μg thymosin β4 (200 μM) with 120 μg agent (1 mM) and 0.2 U guinea pig transglutaminase at room temperature in 240 μl buffer consisting of 10 mM Tris-HCl, pH 7.4, 15 mM CaCl₂, 3 mM DTT. After 1 and 2 hours, 5 μl of the reaction mixture is subjected to HPLC analysis. The reaction is stopped after 4 hours by addition of 5 μl trifluoroacetic acid (TFA). Then the reaction mixture is subjected to preparative HPLC. Separated peptides are concentrated in vacuo and then characterised by amino acid analysis and mass spectrometry.

Proteolytic fragments of agent-linked thymosin β4 are prepared by the following procedure: 50 μg of peptide is incubated with 20 μU AsnC-endoproteinase in 100 μl reaction buffer (50 mM sodium acetate, pH 5.0, 0.2 mM DTT, 0.2 mM EDTA) for 16 hours at room temperature. Then the reaction is stopped by adding 5 μl 10% TFA and products are separated by preparative HPLC. Prior to analysis by the samples are concentrated in vacuo.

### Microinjection Experiments

Microinjection is performed with an ECET cell injection system (Eppendorf, Hamburg, Germany) consisting of the micromanipulator 5170 and the microinjector 5242 adapted to an Axiovert 100 inverted microscope (Zeiss, Göttingen, Germany). Microinjections are visually controlled by a CCD camera on a TV monitor (SSM 121CE, Sony, Tokyo, Japan). Agent-linked thymosin β4 and crosslinked ADP-ribosylated actin:thymosin β4 complex are injected into the cytoplasm at a concentration of 32 μM and 8.27 μM respectively, in 135 mM KCl, 5 mM Na₂HPO₄, pH 7.2. The injection pressure is between 65 and 80 hPa (1 hPa=0.1 kPa) and the injection time between 0.5 and 0.7 seconds.

Agent-linked thymosin β4 is microinjected into the cytoplasm of cells. Directly after microinjection the linked peptide is evenly distributed throughout the cytoplasm. After incubation for 1 hour a pronounced localization the cell nucleus is detected. The N-terminal portion of thymosin β4 contains a sequence stretch enriched in lysine residues (¹⁴KSKLKK¹⁹ (SEQ ID NO:3)) which may be a functional nuclear localisation signal. An N-terminal fragment (thymosin β²⁷⁻⁴³4) containing the ¹⁴KSKLKK¹⁹ (SEQ ID NO:3) sequence exhibits a pronounced nuclear localisation.

1. A pharmaceutically acceptable composition for entering a cell nucleus, comprising a cell nucleus-entering polypeptide comprising at least one of amino acid sequence LKKTET (SEQ ID NO:1), amino acid sequence LKKTNT (SEQ ID NO:2) or amino acid sequence KSKLKK (SEQ ID NO:3), KLKKTET (SEQ ID NO:4), LKKTETQ (SEQ ID NO:5), Thymosin Γ4 (TΓ4), an N-terminal variant of TΓ4, a nucleus-entering C-terminal variant of TΓ4, an N-terminal fragment of T134, an isoform of TΓ4, a splice-variant of TΓ4, oxidized TΓ4, TΓ4 sulfoxide, lymphoid TΓ4, pegylated TΓ4 or a conservative variant thereof, Tβ4^{ala}, Tβ9, Tβ10, Tβ11, Tβ12, Tβ13, Tβ14, Tβ15, gelsolin, vitamin D binding protein (DBP), profilin, cofilin, adsevertin, propomyosin, fincilin, depactin, Dnasel, villin, fragmin, severin, capping protein, β-actinin or acumentin, said polypeptide being linked to a physiologically active agent having at least one of therapeutic or diagnostic application in said cell nucleus.

2. The composition of claim 1, wherein said physiologically active agent comprises a drug, chemotherapeutic agent or nucleic acid sequence.

3. The composition of claim 1 wherein said cell nucleus-entering polypeptide comprises amino acid sequence LKKTET (SEQ ID NO:1) or amino acid sequence KSKLKK (SEQ ID NO:3).

4. The composition of claim 1 wherein said cell nucleus-entering polypeptide comprises thymosin beta 4.

5. The composition of claim 1 wherein said polypeptide comprises an N-terminal fragment of thymosin beta 4.

6. The composition of claim 1 wherein said cell-entering polypeptide linked to said agent is present in a pharmaceutically acceptable carrier.

7. The composition of claim 6 wherein said cell nucleus-entering polypeptide linked to said agent is present in said carrier in concentration within a range of about 0.0001-10% by weight of said carrier.


SEQUENCE LISTING (7 sequences)

SEQ ID NO: 1 - protein, 6 aa, Sus scrofa
LKKTET

SEQ ID NO: 2 - protein, 6 aa, Sus scrofa
LKKTNT

SEQ ID NO: 3 - protein, 6 aa, Sus scrofa
KSKLKK

SEQ ID NO: 4 - protein, 7 aa, Sus scrofa
KLKKTET

SEQ ID NO: 5 - protein, 7 aa, Sus scrofa
LKKTETQ

SEQ ID NO: 6 - protein, 4 aa, Sus scrofa
  MOD_RES (1)..(1): ACETYLATION
SDKP

SEQ ID NO: 7 - protein, 4 aa, Sus scrofa
  MOD_RES (1)..(1): ACETYLATION
SQNY